(19) United States
(12) Patent Application Publication
(10) Pub. No.: US 2019/0164646 A1
(43) Pub. Date: May 30, 2019

(54) METHODS, SYSTEMS, APPARATUSES AND DEVICES FOR FACILITATING HEALTH CARE MANAGEMENT

(71) Applicant: Steven Merahn, Orchard Lake, MI (US)

(72) Inventor: Steven Merahn, Orchard Lake, MI (US)

(21) Appl. No.: 16/204,547

(22) Filed: Nov. 29, 2018

Related U.S. Application Data
(60) Provisional application No. 62/592,077, filed on Nov. 29, 2017.

(51) Int. Cl. G16H 50/20; G16H 10/60; G06F 16/2455

(52) U.S. Cl. CPC G16H 50/20; G06F 16/2455; G16H 10/60

(57) ABSTRACT

Disclosed herein is a system for facilitating health care management. Accordingly, the system may include a communication device configured for transmitting at least one health assessment form to a user device. Further, the communication device may be configured for receiving health assessment data associated with an individual from the user device. Further, the communication device may be configured for transmitting at least one suggested health care plan to the user device. Further, the communication device may be configured for receiving a selection from the user device. Further, the selection corresponds to at least one selected health care plan of the at least one suggested health care plan. Further, the system may include a processing device configured for analyzing the health assessment data. Further, the processing device may be configured for generating the at least one suggested health care plan for the individual based on the analyzing.

The current application claims a priority to the U.S. Provisional Patent application Ser. No. 62/592,077 filed on Nov. 29, 2017.

### TECHNICAL FIELD

Generally, the present disclosure relates to the field of data processing. More specifically, the present disclosure relates to methods, systems, apparatuses and devices for facilitating health care management.

### BACKGROUND

The profession of medicine does not have a standardized framework for providing care plans. Traditionally, each physician maintains a problem list and prescribes medicine or surgery orders for a patient depending on their own decision-making. If the patient has multiple conditions and different physicians are involved in their care, each provider develops their own condition-specific care plan. While electronic health records may contain or aggregate the work of these individual physicians, they are not presented to the patient as a cohesive or coordinated plan of care.

Further, in standard health practice, a physician may not provide the patient with various options based on health of the patient. Therefore, the patient may only know about a limited (and/or only one) number of treatments applicable for the patient. Consequently, the patient may not have any other option to choose as a clinical strategy for the treatment. Accordingly, they resign their fate to the treatment plan suggested by the physician.

Further, with the increase in a number of patients with multiple conditions, a new level of complexity may arise, as each condition may not be managed in isolation from the others. Although each individual condition may be well understood with established treatments, multiple simultaneous conditions interact to present new levels of complexity, including, but not limited to, drug-drug and drug-lab test interactions, in a way that may put providers in a situation where they must prioritize conditions and treatments that may have beneficial effects for some conditions but detrimental effects upon others, requiring outcomes-oriented stratification of an individual patients total management plan.

Further, there may be few existing protocols for handling combinations of simultaneous conditions. However, care providers may not always have immediate knowledge of the appropriate responses to a patient's situation. The care teams may likely to be even larger and more diverse for older patients presenting multiple conditions. Every addition to the care team introduces the opportunity for error in the communications between the team members, as well as the potential for conflicts or redundancies in the decisions made by individual members of the patients “healthcare resource-community”.

Therefore, there is a need for improved methods, systems, apparatuses and devices for facilitating health care management that may overcome one or more of the above-mentioned problems and/or limitations.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method of facilitating health care management, in accordance with some embodiments. Accordingly, the method may include transmitting, using a communication device, at least one health assessment form to a user device. Further, the method may include receiving, using the communication device, at least one health assessment data associated with an individual from the user device. Further, the method may include analyzing, using a processing device, the at least one health assessment data. Further, the method may include generating, using the processing device, at least one suggested health care plan for the individual based on the analyzing. Further, the method may include transmitting, using the communication device, the at least one suggested health care plan to the user device. Further, the method may include receiving, using the communication device, a selection from the user device.

Further disclosed herein is a system for facilitating health care management, in accordance with some embodiments. Accordingly, the system may include a communication device configured for transmitting at least one health assessment form to a user device. Further, the communication device may be configured for receiving at least one health assessment data associated with an individual from the user device. Further, the communication device may be configured for transmitting at least one suggested health care plan to the user device. Further, the communication device may be configured for receiving a selection from the user device. Further, the selection corresponds to at least one selected health care plan of the at least one suggested health care plan. Further, the system may include a processing device configured for analyzing the at least one health assessment data. Further, the processing device may be configured for generating the at least one suggested health care plan for the individual based on the analyzing.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 illustrates a block diagram representation of a system for facilitating health care management, in accordance with some embodiments.

FIG. 3 illustrates a block diagram representation of the system that may further include a storage device configured for retrieving evidence data based on the at least one health assessment data, in accordance with some embodiment.

FIG. 4 illustrates a flowchart of a method to facilitate health care management, in accordance with some embodiment.

FIG. 5 illustrates a flowchart of a method to facilitate storing the evidence data, in accordance with some embodiments.

FIG. 6 illustrates a flowchart of a method to obtain a response from at least one practitioner, in accordance with some embodiment.

FIG. 7 illustrates a flowchart of a method to obtain at least one modified health care plane from the at least one practitioner response and the at least one selected health care plan, in accordance with some embodiments.

FIG. 8 illustrates a flowchart of a method to obtain a clinical data corresponding to the individual, in accordance with some embodiments.

FIG. 9 illustrates a flowchart of a method to facilitate developing evidence-based care plan specific to a user, in accordance with some embodiment.

FIG. 10 illustrates a flowchart of a method to evaluate the health risk assessment, in accordance with some embodiment.

FIG. 11 illustrates a flowchart of a method of providing the evidence-based care plan to a health care professional of the user.

FIG. 12 is a block diagram of a computing device for implementing the methods disclosed herein, in accordance with some embodiments.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of health care management, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.)

corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

According to some embodiments, the present disclosure provides a system and method for patients to create their own plans of care by accessing a database that presents evidence-based solutions that may be parsed by an analysis of patient-level data that may be used to generate a list of individualized risks, conditions and other strategies for health improvement. Further, the disclosed patient driven system may deliver the plan to the physician for review and mutually agreed upon action.

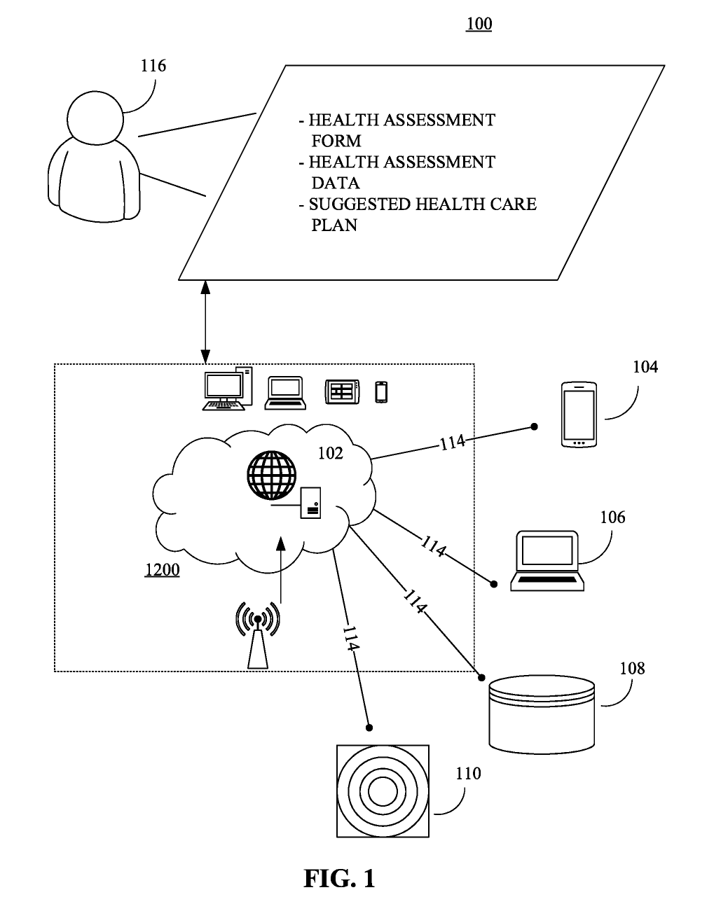

FIG. 1 is an illustration of an online platform 100 consistent with various embodiments of the present disclosure. By way of non-limiting example, the online platform 100 to facilitate health care management may be hosted on a centralized server 102, such as, for example, a cloud computing service. The centralized server 102 may communicate with other network entities, such as, for example, a mobile device 104 (such as a smartphone, a laptop, a tablet computer etc.), other electronic devices 106 (such as desktop computers, server computers etc.), databases 108, and sensors 110 over a communication network 114, such as, but not limited to, the Internet. Further, users of the online platform 100 may include relevant parties such as, but not limited to, end users, administrators, service providers, service consumers and so on. Accordingly, in some instances, electronic devices operated by the one or more relevant parties may be in communication with the platform.

A user 116, such as the one or more relevant parties, may access online platform 100 through a web based software application or browser. The web based software application may be embodied as, for example, but not be limited to, a website, a web application, a desktop application, and a mobile application compatible with a computing device X00.

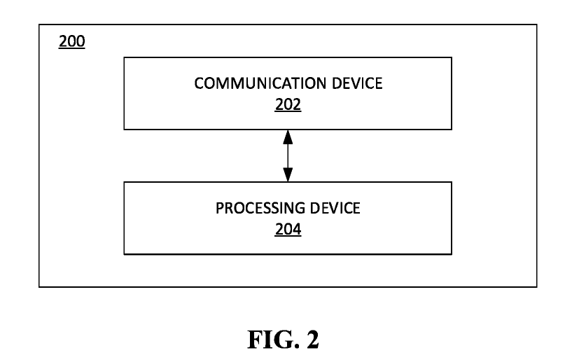

FIG. 2 illustrates a block diagram representation of a system 200 for facilitating health care management, in accordance with some embodiments. The system 200 may include a communication device 202 configured for transmitting at least one health assessment form to a user device. Further, the at least one health assessment form may be a digital form. For instance, the health assessment form may include, but not limited to, a web-based form. A user may use the at least one health assessment form to share health related information. Further, the communication device 202 may be configured for receiving at least one health assessment data associated with an individual from the user device. The at least one health assessment data, in an instance, may be any data related to the health associated with the user. In some embodiments, the at least one health assessment data may include at least one medical condition and at least one health risk factor. In some embodiments, the health assessment data may include sensor data associated with at least one sensor configured for sensing at least one health related variable corresponding to the individual. Further, the user device may include the at least one sensor. Further, the user device, in an instance, may be any IoT based device that may be configured to communicate with the system 200. Further, the user device, in an instance, may include, but not limited to, a smartphone, a smartwatch, a PC, a laptop etc. Further, the at least one sensor, in an instance, may be embedded within the user device. Further, the at least one sensor, in an instance, may include, but not limited to, heart rate sensors, pressure sensors, temperature sensors etc. Further, the communication device 202 may be configured for transmitting at least one suggested health care plan to the user device. Further, the at least one suggested health care plan, in an instance, may be a treatment plan that may be followed by the user to achieve a specific goal. Further, the at least one suggested health care plan may include at least one clinical strategy and at least one health goal corresponding to the at least one medical condition. In some embodiments, the at least one suggested health care plan may include a natural language description of the at least one clinical strategy and the at least one health goal. Further, the communication device 202 may be configured for receiving a selection from the user device. Further, the selection corresponds to at least one selected health care plan of the at least one suggested health care plan. Further, the system 200 may include a processing device 204 configured for analyzing the at least one health assessment data. Further, the processing device 204 may be configured for generating the at least one suggested health care plan for the individual based on the analyzing.

In some embodiments, the communication device 202 may be further configured for transmitting the at least one selected health care plan and the health assessment data to at least one practitioner device. Further, the at least one practitioner device may be any device that may be operated by a health care practitioner. Further, the communication device 202 may be configured for receiving at least one practitioner response from the at least one practitioner device. Further, the at least one practitioner response may be any reply and/or a feedback from the at least one practitioner device. In an instance, the at least one practitioner response may include a recommendation to change the dosage prescribed in the at least one selected health care plan. Further, the communication device 202 may be configured for transmitting the at least one practitioner response to the user device. Further, in some embodiments, the system 200 may deliver the at least one suggested health care plan to the at least one practitioner device (such as a device that may be used by a physician) for review and/or mutually agreed upon the at least one suggested health care plan.

In some embodiments, the processing device 204 may be further configured for generating at least one modified health care plan based on the at least one practitioner response and the at least one selected health care plan. Further, the modified health care plan, in an instance, may be the at least one health care plan that may be altered by the system 200 based on the at least one practitioner response. For example, the dosage prescribed in the at least one selected health care plan may be modified based on the at least one practitioner response. Further, the communication device 202 may be configured for transmitting the at least one modified health care plan to the user device.

In some embodiments, the communication device 202 may be further configured for transmitting a user identifier associated with the individual to an electronic medical record (EMR) database. Accordingly, the user identifier, in an instance, may be any variable that may reflect an identity of the user. For instance, the user identifier may include, but not limited to, a social security number or a mobile phone number associated with the user. Further, the communication device 202 may be configured for receiving clinical data corresponding to the individual from the EMR database. Further, the clinical data, in an instance, may include, but not limited to, medical test reports associated with the user. In some embodiments, the clinical data may include a plurality of medical conditions associated with the individual and a plurality of clinical interventions associated with the plurality of medical conditions. Further, the plurality of clinical interventions, in an instance, may include, but not limited to, medical treatments that may be carried out to improve, maintain, or assess the health associated with the user. Further, the processing device 204 may be configured for analyzing the clinical data. Further, the analyzing may include determining one or more of at least one condition interaction between two or more medical conditions of the plurality of medical conditions and at least one intervention interaction between two or more clinical interventions of the plurality of clinical interventions. Further, the generating of the at least one suggested health care plan may be based on the analyzing of the clinical data. Further, the generating of the at least one suggested health care plan may be based on one or more of the at least one condition interaction and the at least one intervention interaction.

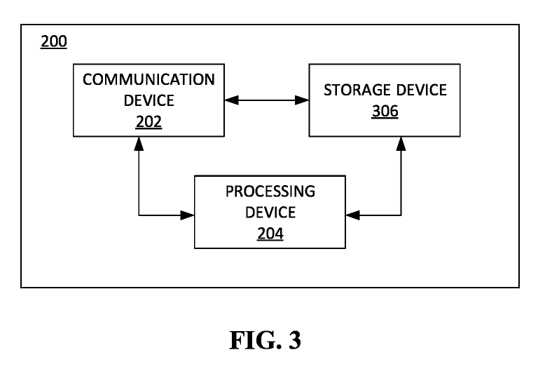

FIG. 3 illustrates a block diagram representation of the system 200 that may further include a storage device 306 configured for retrieving evidence data based on the at least one health assessment data, in accordance with some embodiment. Further, the generating of the at least one suggested health care plan may be further based on the evidence data. Further, in some embodiments, the processing device 204 may be configured to create the at least one suggested health care plan by accessing the storage device 306 that may present evidence data. In some embodiments, the processing device 204 may be further configured for generating at least one search query based on the at least one health assessment data. Further, the at least one search query, in an instance, may be any health related query such as (but not limited to) a disease name, symptoms associated with a disease etc. Further, the processing device 204 may be configured for analyzing at least one search result. Further, the processing device 204 may be configured for generating the evidence data based on the analyzing of the at least one search result. Further, the communication device 202 may be further configured for transmitting the at least one search query to at least one external database. Further, the at least one external database, in an instance, may be storage space that may be configured to store data (such as clinical data, test results, medical studies etc.) in an organized form. For example, the at least one external database may be related to a service such as WebMD.com™. Further, the communication device 202 may be configured for receiving at least one search result from the at least one external database. Further, the at least one search result, in an instance, may be any data that may be extracted from the at least one external database based on the at least one search query. Further, the storage device 306 may be further configured for storing the evidence data.

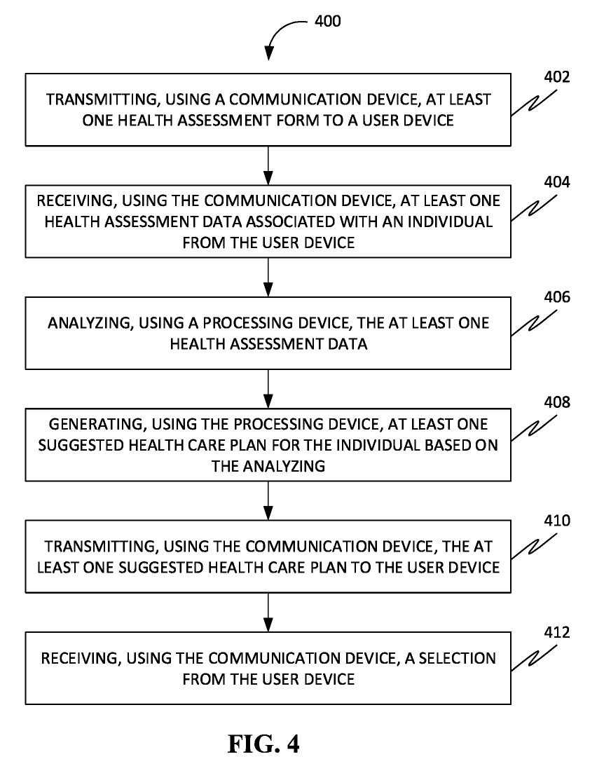

FIG. 4 illustrates a flowchart of a method 400 to facilitate health care management, in accordance with some embodiment. At 402, the method 400 may include transmitting, using a communication device (such as the communication device 202), at least one health assessment form to a user device. Further, at 404, the method 400 may include receiving, using the communication device, at least one health assessment data associated with an individual from the user device. In some embodiments, the at least one health assessment data may include at least one medical condition and at least one health risk factor. In some embodiments, the at least one health assessment data may include sensor data associated with at least one sensor configured for sensing at least one health related variable corresponding to the individual. Further, the user device may include the at least one sensor.

Further, at 406, the method 400 may include analyzing, using a processing device (such as the processing device 204), the at least one health assessment data. Further, at 408, the method 400 may include generating, using the processing device, at least one suggested health care plan for the individual based on the analyzing. Further, the at least one suggested health care plan may include at least one clinical strategy and at least one health goal corresponding to the at least one medical condition. In some embodiments, the at least one suggested health care plan may include a natural language description of the at least one clinical strategy and the at least one health goal. Further, at 410, the method 400 may include transmitting, using the communication device, the at least one suggested health care plan to the user device. Further, at 412, the method 400 may include receiving, using the communication device, a selection from the user device. Further, the selection corresponds to at least one selected health care plan of the at least one suggested health care plan.

Further, in some embodiments, the method 400 may include retrieving, using a storage device (such as the storage device 306), evidence data based on the at least one health assessment data. Further, the generating of the at least one suggested health care plan may be based on the evidence data.

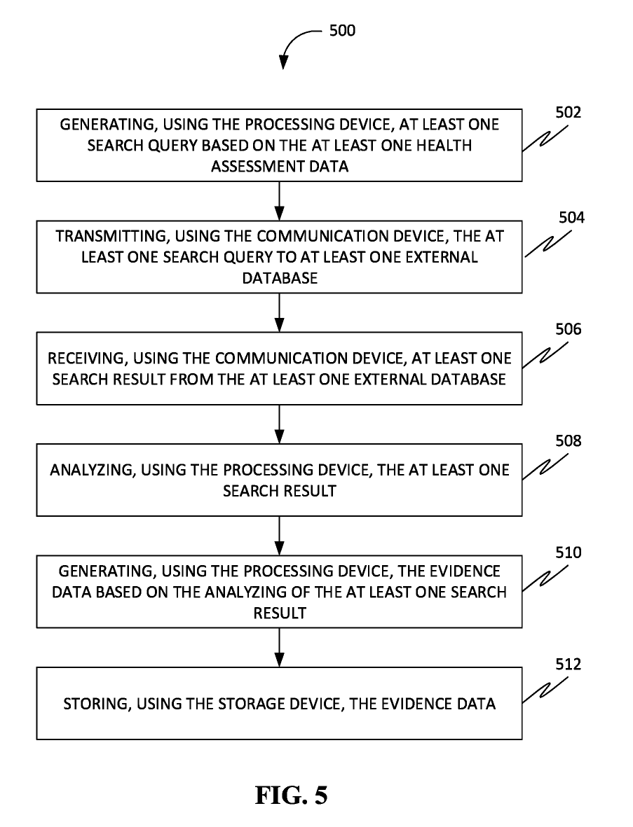

FIG. 5 illustrates a flowchart of a method 500 to facilitate storing the evidence data, in accordance with some embodiments. Further, at 502, the method 500 may include generating, using the processing device, at least one search query based on the at least one health assessment data. Further, at 504, the method 500 may include transmitting, using the communication device, the at least one search query to at least one external database. Further, at 506, the method 500 may include receiving, using the communication device, at least one search result from the at least one external database. Further, at 508, the method 500 may include analyzing, using the processing device, the at least one search result. Further, at 510, the method 500 may include generating, using the processing device, the evidence data based on the analyzing of the at least one search result. Further, at 512, the method 500 may include storing, using the storage device, the evidence data.

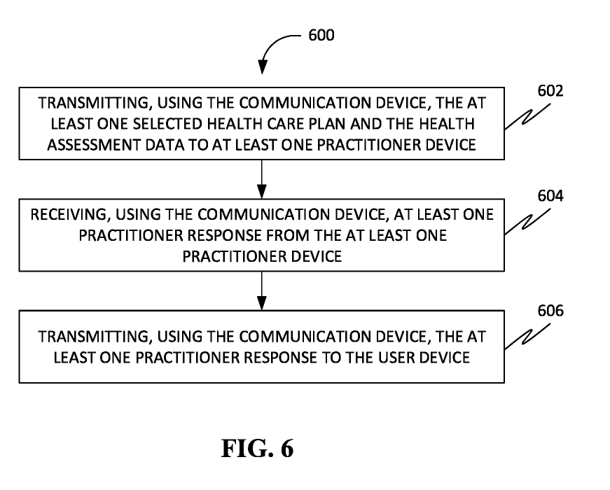

FIG. 6 illustrates a flowchart of a method 600 to obtain a response from at least one practitioner, in accordance with some embodiment. At 602, the method 600 may include transmitting, using the communication device, the at least one selected health care plan and the health assessment data to at least one practitioner device. Further, at 604, the method 600 may include receiving, using the communication device, at least one practitioner response from the at least one practitioner device. Further, at 606, the method 600 may include transmitting, using the communication device, the at least one practitioner response to the user device.

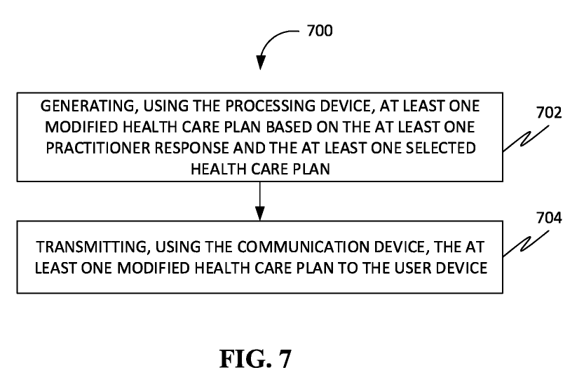

FIG. 7 illustrates a flowchart of a method 700 to obtain at least one modified health care plane from the at least one practitioner response and the at least one selected health care plan, in accordance with some embodiments. Further, the modified health care plan, in an instance, may be the at least one health care plan that may be altered by the online platform 100 based on the at least one practitioner response. At 702, the method 700 may include generating, using the processing device, at least one modified health care plan based on the at least one practitioner response and the at least one selected health care plan. Further, at 704, the method 700 may include transmitting, using the communication device, the at least one modified health care plan to the user device.

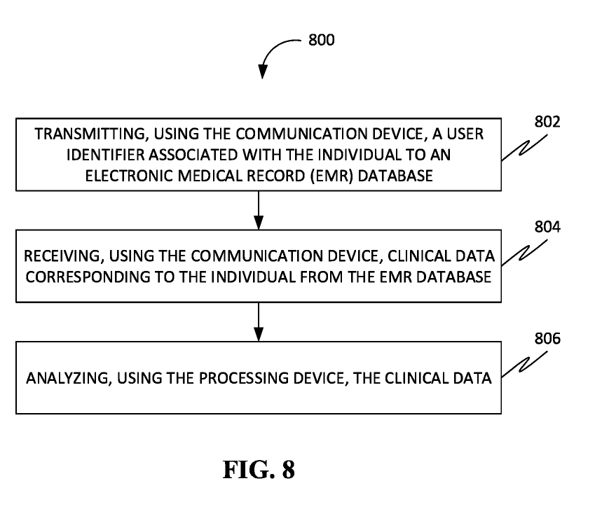

FIG. 8 illustrates a flowchart of a method 800 to obtain a clinical data corresponding to the individual, in accordance with some embodiments. Further, at 802, the method 800 may include transmitting, using the communication device, a user identifier associated with the individual to an electronic medical record (EMR) database. Further, at 804, the method 800 may include receiving, using the communication device, clinical data corresponding to the individual from the EMR database. In some embodiments, the clinical data may include a plurality of medical conditions associated with the individual and a plurality of clinical interventions associated with the plurality of medical conditions. Further, at 806, the method 800 may include analyzing, using the processing device, the clinical data. Further, the generating of the at least one suggested health care plan may be based on the analyzing of the clinical data. Further, the analyzing may include determining one or more of at least one condition interaction between two or more medical conditions of the plurality of medical conditions and at least one intervention interaction between two or more clinical interventions of the plurality of clinical interventions. Further, the generating of the at least one suggested health care plan may be further based on one or more of the at least one condition interaction and the at least one intervention interaction.

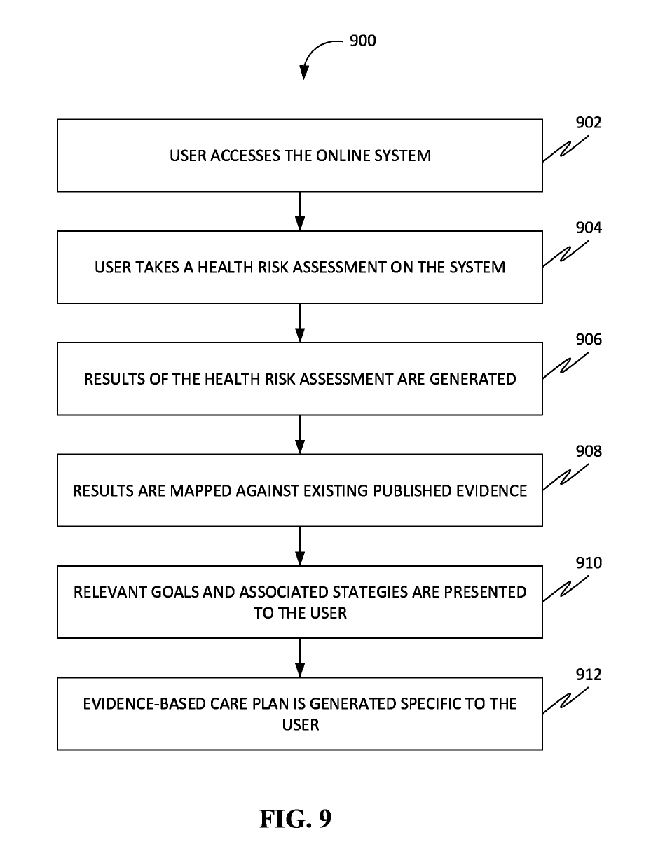

FIG. 9 illustrates a flowchart of a method 900 to facilitate developing evidence-based care plan specific to a user, in accordance with some embodiment. Accordingly, at 902, the method 900 may include accessing an online system by the user. Further, the online system may be configured to be accessible by any computing device such as a personal computer or mobile device. In another embodiment, the online system may be a mobile application specifically for mobile devices. When the online system may be accessed by the user, the user may be prompted to create a user profile and providing basic information such as the user's name, and/or email address etc. Further, at 904, the method 900 may include completing a health risk assessment on the online system. Once the user profile may be created by the user, the user may be prompted to complete the health risk assessment and/or given an option to choose another standard health risk assessment. Further, the health risk assessment may include standard questions for the user such as (but not limited to) the weight of the user, the height of the user, and/or waist size of the user etc. Further, the health risk assessment may additionally include questions about the user's social status, behavioral status, medications, health conditions, medications, lifestyle, state of wellness, and demographic information of the user. All the information gathered of the user may be stored within a database of the online system. Further, at 906, the method 900 may include generating results of the health risk assessment. Further, at 908, the method 900 may include mapping the results against existing published evidence. Further, at 910, the method 900 may include presenting a number of relevant goals and associated strategies to the user. Further, at 912, the method 900 may include generating evidence-based care plan specific to the user.

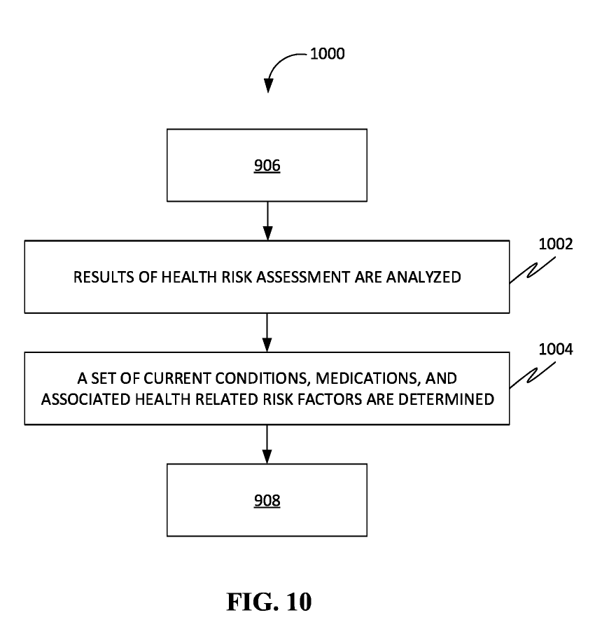

FIG. 10 illustrates a flowchart of a method 1000 to evaluate the health risk assessment, in accordance with some embodiment. Accordingly, at 1002, the method 1000 may include analyzing results of the health risk assessment. Further, at 1004, the method 1000 may include determining a set of current conditions, medications, and associated health related risk factors. Further, the information (and/or responses) gathered of the user from the health risk assessment may be analyzed by the online system and the set of current conditions, medications, and associated health risk factors, specific to the user, may be determined. For example, the information gathered of the user from the health risk assessment may provide that the user has diabetes and therefore the user has a higher risk for another condition such as heart disease. Further, the results of the health risk assessment may be mapped against existing published evidence, third-party quality measures, and other applicable best practices and clinical order sets.

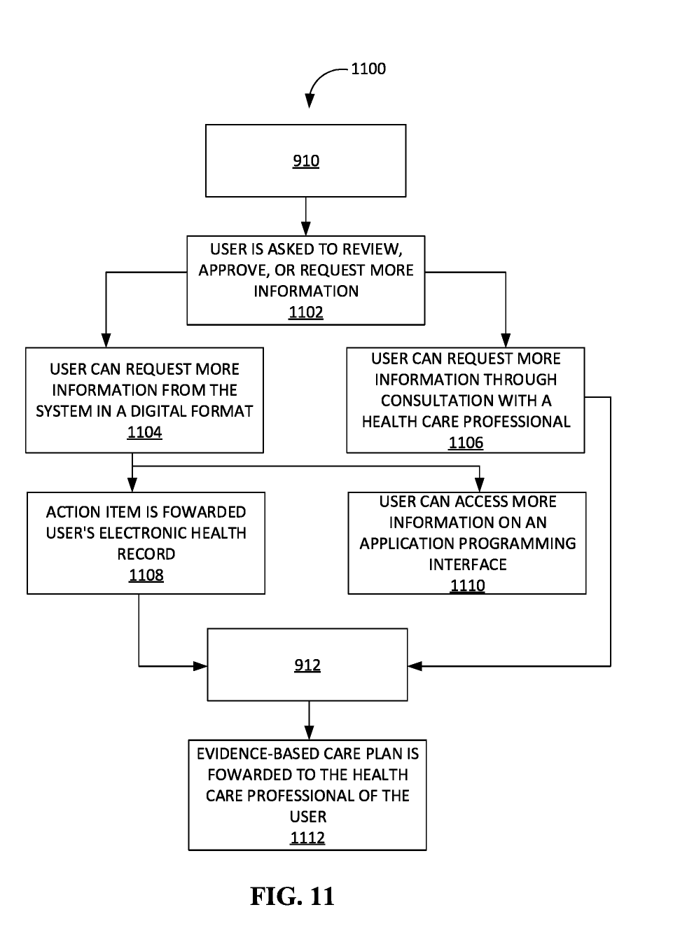

FIG. 11 illustrates a flowchart of a method 1100 to sharing the evidence-based care plan with a health care professional (such as a physician) of the user. Accordingly, a number of relevant goals and associated clinical strategies may be categorized and may be presented to the user in a proprietary health literacy rubric after the results of the health risk assessment may be mapped. For example, if the user suffers from sleep apnea, a relevant goal may be for the user to lose weight. In standard health practice, a health care professional may not provide the user with various options based on results gathered on the user and therefore the user may only know about one relevant goal for treatment. Further, at 1102, the method 1100 may include asking the user to review, approve, and/or request more information. For instance, the online system may prompt the user to review, approve, or request more information for each relevant goal and associated clinical strategy. Further, an output of the user's decision making may be a patient generated evidence-based care plan in an information architecture based on categorizing care plan goals into six categories. Further, the user may decide which parts of the generated evidence-based care plan to commit to such as particular relevant goals. Further, at 1104, the method 1100 may include requesting more information from the online system in a digital format. Further, at 1106, the method 1100 may include requesting more information through consultation with the health care professional. For instance, if the user wants more information, the user may request more information from the online system or select an option for consultation with his or her health care professional (such as the physician). Further, at 1108, the method 1100 may include forwarding an action item. Further, at 1110, the method 1100 may include accessing more information on an application programming interface. For instance, if the user requests more information from the online system, the action item may be forwarded to either the user's electronic health record, so the requests may be fulfilled according to meaningful use standard for patient education materials or to another system by which information and material may be delivered to the user such as the application programming interface containing the electronic health record of the user. Further, at 1112, the method 1100 may include forwarding the evidence-based care plan to the health care professional of the user. For instance, an overall care plan chosen by the user may be forwarded to the user's health care professional to review, follow-up on the requests for more information and generation of order to fulfill the patients requested clinical strategies. Further, in some embodiments, the user may deliver the best care plan to the physician for review and/or may mutually agree upon action.

The disclosed systems and methods may allow the user to decide the best care plan for himself or herself and which goals to commit to rather than being limited to, or solely responsive to, what the health care professional recommends.

Further, the disclosed system and method may increase a level of adherence and affinity for the user's care plan. Further, the disclosed system and method may shift the locus of control from the health care professional to the user. Further, the disclosed system and method may allow the user to go about a care plan which may be more reasonable and achievable based on the user's characteristics which may be analyzed by the system through the information gathered by the health risk assessment.

Although the disclosed systems and methods may have been explained in relation to preferred embodiments, it is to be understood that many other possible modifications and variations may be made without departing from the spirit and scope of the disclosure.

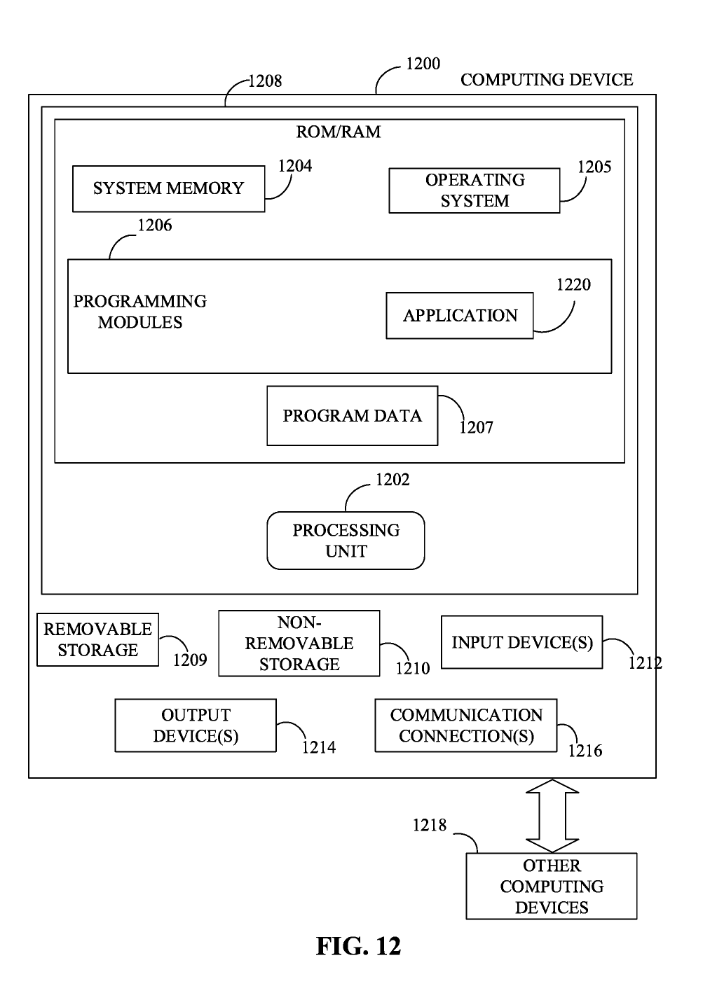

With reference to FIG. 12, a system consistent with an embodiment of the disclosure may include a computing device or cloud service, such as computing device 1200. In a basic configuration, computing device 1200 may include at least one processing unit 1202 and a system memory 1204. Depending on the configuration and type of computing device, system memory 1204 may comprise, but is not limited to, volatile (e.g. random-access memory (RAM)), non-volatile (e.g. read-only memory (ROM)), flash memory, or any combination. System memory 1204 may include operating system 1205, one or more programming modules 1206, and may include a program data 1207. Operating system 1205, for example, may be suitable for controlling computing device 1200's operation. In one embodiment, programming modules 1206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 12 by those components within a dashed line 1208.

Computing device 1200 may have additional features or functionality. For example, computing device 1200 may also include additional data storage devices (removable and/or non-removable) such as, for example, magnetic disks, optical disks, or tape. Such additional storage is illustrated in FIG. 12 by a removable storage 1209 and a non-removable storage 1210. Computer storage media may include volatile and non-volatile, removable and non-removable media implemented in any method or technology for storage of information, such as computer-readable instructions, data structures, program modules, or other data. System memory 1204, removable storage 1209, and non-removable storage 1210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1200. Any such computer storage media may be part of device 1200. Computing device 1200 may also have input device(s) 1212 such as a keyboard, a mouse, a pen, a sound input device, a touch input device, a location sensor, a camera, a biometric sensor, etc. Output device(s) 1214 such as a display, speakers, a printer, etc. may also be included. The aforementioned devices are examples and others may be used.

Computing device 1200 may also contain a communication connection 1216 that may allow device 1200 to communicate with other computing devices 1218, such as over a network in a distributed computing environment, for example, an intranet or the Internet. Communication connection 1216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1204, including operating system 1205. While executing on processing unit 1202, programming modules 1206 (e.g., application 1220 such as a media player) may perform processes including, for example, one or more stages of methods, algorithms, systems, applications, servers, databases as described above. The aforementioned process is an example, and processing unit 1202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. A method of facilitating health care management, the method comprising:
transmitting, using a communication device, at least one health assessment form to a user device;
receiving, using the communication device, at least one health assessment data associated with an individual from the user device;
analyzing, using a processing device, the at least one health assessment data;
generating, using the processing device, at least one suggested health care plan for the individual based on the analyzing;
transmitting, using the communication device, the at least one suggested health care plan to the user device; and
receiving, using the communication device, a selection from the user device, wherein the selection corresponds to at least one selected health care plan of the at least one suggested health care plan.

2. The method of claim 1 further comprising:
transmitting, using the communication device, the at least one selected health care plan and the at least one health assessment data to at least one practitioner device;
receiving, using the communication device, at least one practitioner response from the at least one practitioner device; and
transmitting, using the communication device, the at least one practitioner response to the user device.

3. The method of claim 2 further comprising:
generating, using the processing device, at least one modified health care plan based on the at least one practitioner response and the at least one selected health care plan; and
transmitting, using the communication device, the at least one modified health care plan to the user device.

4. The method of claim 1 further comprising retrieving, using a storage device, evidence data based on the at least one health assessment data, wherein the generating of the at least one suggested health care plan is further based on t evidence data.

5. The method of claim 4 further comprising:
generating, using the processing device, at east one search query based on the at least one health assessment data;
transmitting, using the communication device, the at least one search query to at least one external database;
receiving, using the communication device, at least one search result from the at least one external database;
analyzing, using the processing device, the at least one search result;
generating, using the processing device, the evidence data based on the analyzing of the at least one search result; and
storing, using the storage device, the evidence data.

6. The method of claim 1 further comprising:
transmitting, using the communication device, a user identifier associated with the individual to an electronic medical record (EMR) database;
receiving, using the communication device, clinical data corresponding to the individual from the EMR database; and
analyzing, using the processing device, the clinical data, wherein the generating of the at least one suggested health care plan is further based on the analyzing of the clinical data.

7. The method of claim 6 wherein, the clinical data comprises a plurality of medical conditions associated with the individual and a plurality of clinical interventions associated with the plurality of medical conditions, wherein the analyzing comprises determining at least one of at least one condition interaction between two or more medical conditions of the plurality of medical conditions and at least one intervention interaction between two or more clinical interventions of the plurality of clinical interventions, wherein the generating of the at least one suggested health care plan is further based on at least one of the at least one condition interaction and the at least one intervention interaction.

8. The method of claim 1, wherein the at least one health assessment data comprises at least one medical condition and at least one health risk factor, wherein the at least one suggested health care plan comprises at least one clinical strategy and at least one health goal corresponding to the at least one medical condition.

9. The method of claim 8, wherein the at least one suggested health care plan comprises a natural language description of the at least one clinical strategy and the at least one health goal.

10. The method of claim 1, wherein the at least one health assessment data comprises sensor data associated with at least one sensor configured for sensing at least one health related variable corresponding to the individual, wherein the user device comprises the at least one sensor.

11. A system for facilitating health care management, the system comprising:
a communication device configured for:
transmitting at least one health assessment form to a user device;
receiving at least one health assessment data associated with an individual from the user device;
transmitting at least one suggested health care plan to the user device; and
receiving a selection from the user device, wherein the selection corresponds to at least one selected health care plan of the at least one suggested health care plan; and
a processing device configured for:
analyzing the at least one health assessment data; and
generating the at least one suggested health care plan for the individual based on the analyzing.

12. The system of claim 11, wherein the communication device is further configured for:
transmitting the at least one selected health care plan and the at least one health assessment data to at least one practitioner device;
receiving at least one practitioner response from the at least one practitioner device; and
transmitting the at least one practitioner response to the user device.

13. The system of claim 12, wherein the processing device is further configured for generating at least one modified health care plan based on the at least one practitioner response and the at least one selected health care plan, wherein the communication device is further configured for transmitting the at least one modified health care plan to the user device.

14. The system of claim 11 further comprising a storage device configured for retrieving evidence data based on the at least one health assessment data, wherein the generating of the at least one suggested health care plan is further based on the evidence data.

15. The system of claim 14, wherein the processing device is further configured for:
generating at least one search query based on the at least one health assessment data;
analyzing at least one search result; and
generating the evidence data based on the analyzing of the at least one search result, wherein the communication device is further configured for:
transmitting the at least one search query to at least one external database; and
receiving at least one search result from the at least one external database, wherein the storage device is further configured for storing the evidence data.

16. The system of claim 11, wherein the communication device is further configured for:
transmitting a user identifier associated with the individual to an electronic medical record (EMR) database; and
receiving clinical data corresponding to the individual from the EMR database, wherein the processing device is further configured for analyzing the clinical data, wherein the generating of the at least one suggested health care plan is further based on the analyzing of the clinical data.

17. The system of claim 16 wherein, the clinical data comprises a plurality of medical conditions associated with the individual and a plurality of clinical interventions associated with the plurality of medical conditions, wherein the analyzing comprises determining at least one of at least one condition interaction between two or more medical conditions of the plurality of medical conditions and at least one intervention interaction between two or more clinical interventions of the plurality of clinical interventions, wherein the generating of the at least one suggested health care plan is further based on at least one of the at least one condition interaction and the at least one intervention interaction.

18. The system of claim 11, wherein the at least one health assessment data comprises at least one medical condition and at least one health risk factor, wherein the at least one suggested health care plan comprises at least one clinical strategy and at least one health goal corresponding to the at least one medical condition.

19. The system of claim 18, wherein the at least one suggested health care plan comprises a natural language description of the at least one clinical strategy and the at least one health goal.

20. The system of claim 11, wherein the at east one health assessment data comprises sensor data associated with at least one sensor configured for sensing at least one health related variable corresponding to the individual, wherein the user device comprises the at least one sensor.
